(19) United States
(12) Patent Application Publication
(10) Pub. No.: US 2003/0049606 A1
(43) Pub. Date: Mar. 13, 2003

(54) Novel anti-viral and anti-proliferative agents derived from STAT1 transcription factor

(76) Inventors: Antonis E. Koromilas, Montreal (CA); Andrew Hoi-Tao Wong, Brooklyn, NY (US)

Correspondence Address: Ogilvy Renault, Suite 1600, 1981 McGill College Avenue, Montreal, QC, H3A 2Y3, CA

(21) Appl. No.: 10/054,965

(22) Filed: Jan. 25, 2002

Related U.S. Application Data
(60) Provisional application No. 60/317,922, filed on Sep. 10, 2001.

(51) Int. Cl. C12Q 1/70; A61K 39/395; A61K 39/42; C07K 2/00; C07K 4/00; C07K 5/00; C07K 7/00; C07K 14/00; C07K 16/00; C07K 17/00; A61K 38/00; C12P 21/08

(52) U.S. Cl. 435/5; 424/143.1; 424/159.1; 530/388.7; 530/300

(57) ABSTRACT

A STAT1 mutant which is unable to bind PKR is provided. The mutant is useful to enhance endogenous anti-viral and anti-proliferative activity in mammals.

### RELATED APPLICATIONS

This application claims priority from U.S. Provisional Patent Application Serial No. 60/317,922 filed Sep. 10, 2001.

### FIELD OF THE INVENTION

The present invention relates to mutants derived from the transcription factor, STAT1 (Signal Transducer and Activator of Transcription1), which enhance endogenous anti-viral and anti-proliferative activity. In particular, the present invention relates to anti-viral and anti-proliferative mutants of STAT1 which do not bind double-stranded RNA-activated protein kinase (PKR).

### BACKGROUND OF THE INVENTION

Cytokines and growth factors exert a diverse range of biological activities, from host defense, growth regulation, to immuno-modulation. Upon ligand binding to cell-surface receptors, JAK¹ kinases are activated and proceed to phosphorylate the receptor on tyrosine residues, which then function as docking sites for cytoplasmic transcription factors of the STAT family (Leonard, W. J. et al. (1998) Annu. Rev. Immunol. 16, 293-322; Stark, G. R., et al. (1998) Annu. Rev. Biochem. 67, 227-264). STATs are subsequently activated by tyrosine phosphorylation, dimerize by phosphotyrosyl. SH2 interactions, and translocate to the nucleus to induce transcription of cytokine-responsive genes (Darnell, J. E., Jr. (1997) Science 277, 1630-1635). A single tyrosine phosphorylation site in the carboxyl-terminal activation domain is absolutely essential for STAT dimerization and DNA binding (Darnell, J. E., Jr. (1997) Science 277, 1630-1635), whereas phosphorylation of a serine residue in this region is important for transactivational activity (Decker, T., et al. (2000) Oncogene 19, 2628-2637).

One of the major STATs intimately involved in both the innate and acquired immune responses is STAT1. Upon virus infection or exposure to interferons (IFNs), STAT1 is found in protein complexes that bind specific DNA sequences upstream to genes responsible for host resistance. For instance, IFN-a/b induces formation of the heterodimeric ISGF3, whereas IFN-y induces binding of homodimeric STAT1 (Stark, G. R., et al. (1998) Annu. Rev. Biochem. 67, 227-264; Darnell, J. E., Jr. (1997) Science 277, 1630-1635). Moreover, dsRNA, an intermediate produced during virus replication, can also activate STAT1 DNA binding (Bandyopadhyay, S. K., et al. (1995) J. Biol. Chem. 270, 19624-19629; Wong, A. H., et al. (1997) EMBO J. 16, 1291-1304). The non-redundant role of STAT1 in the antiviral response is further appreciated by findings that stat1 null mice (STAT1^{-/-}) are highly susceptible to microbial infection (Meraz, M. A., et al. (1996) Cell 84, 431-442; Durbin, J. E., et al. (1996) Cell 84, 443-450). IFN signaling leads to the expression of a number of genes, one of which encodes for the dsRNA-dependent protein kinase, PKR (Kaufman, R. J. (2000) in Translational Control of Gene Expression (Sonenberg, N., et al.) pp. 503-527, Cold Spring Harbor Laboratory, Cold Spring Harbor, N.Y.; Clemens, M. J., et al. (1997) J. Interferon Cytokine Res. 17, 503-524). PKR is a serine/threonine protein kinase that displays two distinct activities: (i) autophosphorylation upon dsRNA binding (Kaufman, R. J. (2000) in Translational Control of Gene Expression (Sonenberg, N., et al.) pp. 503-527, Cold Spring Harbor Laboratory, Cold Spring Harbor, N.Y.; Clemens, M. J., et al. (1997) J. Interferon Cytokine Res. 17, 503-524) and (ii) phosphorylation of the eukaryotic translation initiation factor eIF-2a (Kaufman, R. J. (2000) in Translational Control of Gene Expression (Sonenberg, N., Hershey, J. W. B., and Mathews, M. B., eds) pp. 503-527, Cold Spring Harbor Laboratory, Cold Spring Harbor, N.Y.; Clemens, M. J., and Elia, A. (1997) J. Interferon Cytokine Res. 17, 503-524), a modification resulting in inhibition of protein synthesis (Hershey, J. W., (1991) Annu. Rev. Biochem. 60, 717-755). Several studies with cultured cells provide evidence for antiviral (Katze, M. G. (1995) Trends Microbiol. 3, 75-78; Gale, M. J., et al. (1998) Pharmacol. Ther. 78, 29-46), antiproliferative, and tumor suppressor functions of PKR (Kaufman, R. J. (2000) in Translational Control of Gene Expression (Sonenberg, N., Hershey, J. W. B., and Mathews, M. B., eds) pp. 503-527, Cold Spring Harbor Laboratory, Cold Spring Harbor, N.Y.; Clemens, M. J., and Elia, A. (1997) J. Interferon Cytokine Res. 17, 503-524). However, pkr null (PKR^{-/-}) mice exhibit a modest susceptibility to viral infection (Yang. Y. L., et al. (1995) EMBO J. 14, 6095-6106; Abraham, N., et al. (1998) J. Biol. Chem. 274, 5953-5962), and show no signs of tumor formation (Yang. Y. L., et al. (1995) EMBO J. 14, 6095-6106; Abraham, N., et al. (1998) J. Biol. Chem. 274, 5953-5962), suggesting that the lack of PKR can be compensated by other PKR-like molecules (Kaufman, R. J. (2000) in Translational Control of Gene Expression (Sonenberg, N., Hershey, J. W. B., and Mathews, M. B., eds) pp. 503-527, Cold Spring Harbor Laboratory, Cold Spring Harbor, N.Y.; Clemens, M. J., and Elia, A. (1997) J. Interferon Cytokine Res. 17, 503-524), This hypothesis is supported by the recent identification of the PKR-related genes, PERK/PEK (Ron, D., and Harding, H. P, (2000) in Translational Control of Gene Expression (Sonenberg, N., Hershey, J. W. B., and Mathews, M. B., eds) pp. 547-560, Cold Spring Harbor Laboratory, Cold Springs Harbor, N.Y.) and the mouse homolog of the yeast eIF-2a kinase, GCN2 (Berlanga, J. J., et al. (1999) Eur. J. Biochem. 265, 754-762).

An association between PKR and STAT1 has previously been described. This interaction takes place in unstimulated cells and diminishes upon treatment with IFNs or dsRNA. Increased levels of PKR-STAT1 complex have a negative effect on STAT1 DNA-binding and transactivation capacities, thereby decreasing a host's potential for anti-viral resistance and resistance to uncontrolled cellular proliferation.

It would be desirable, thus, to provide an agent derived from an endogenous cellular compound which enhances natural immune response to malady such as viral infection and uncontrolled proliferation of cells.

### SUMMARY OF THE INVENTION

Accordingly, in one aspect of the present invention, there is provided an anti-viral mutant of STAT1.

In another aspect, the present invention provides an anti-viral composition comprising an anti-viral STAT1 mutant in combination with a pharmaceutically acceptable carrier.

In another aspect of the present invention, there is provided an anti-proliferative composition comprising an anti-proliferative STAT1 mutant in combination with a pharmaceutically acceptable carrier.

In another aspect of the present invention, there is provided a method of treating a viral infection in a mammal comprising the step of administering to the mammal a therapeutically effective amount of an anti-viral STAT1 mutant.

In a further aspect of the present invention, a method of treating a condition associated with the uncontrolled proliferation of cells in a mammal, comprising the step of administering to the mammal a therapeutically effective amount of an anti-proliferative STAT1 mutant.

Embodiments of the present invention will be described in more detail herein by reference to the following drawings in which:

### BRIEF DESCRIPTION OF THE DRAWINGS

FIG. 1 is the nucleic acid sequence of human STAT1 (SEQ ID No: 1);

FIG. 2A identifies the five regions of STAT1;

FIG. 2B illustrates the results of pull-down assays in which GST alone or GST-PKR C was co-incubated with [³⁵S]methionine-labeled, in vitro translated STAT1 proteins and subjected to SDS-PAGE to determine the region of STAT1 that binds PKR;

FIG. 2C identifies regions of STAT1 between amino acid residues 304 to 413 and the results of further pull-down assays in which GST alone or GST-PKR C was incubated with in vitro translated STAT1 proteins truncated from either the amino or carboxyl terminus and subjected to SDS-PAGE;

FIG. 2D illustrates the results of similar pull-down assays using STAT1 proteins further truncated from the carboxyl terminus;

FIG. 2E illustrates the co-precipitation of GST alone or GST-PKR C with in vitro translated STAT1 1-413 wild-type (WT) or STAT1 mutant (TM);

FIG. 2F illustrates the results of cell extracts (U3A cells transfected with HA-STAT1αWT or TM in the presence or absence of PKRK296R) immunoprecipitated with anti-PKR antibodies and immunoblotted with HA (top panel) and PKR antibodies (bottom panel);

FIGS. 3A-E illustrates experimental results in which STAT1 TM was found to display elevated transcriptional properties; and

FIGS. 4A-B illustrates experimental results by which STAT1 TM was determined to possess enhanced anti-proliferative and anti-viral properties.

### DETAILED DESCRIPTION OF THE INVENTION

An anti-viral STAT1 mutant is provided. The mutant is produced by altering the sequence in the PKR binding region of STAT1 that results in the loss of PKR binding ability. The STAT1 mutant has also been shown to have anti-proliferative activity.

The term “anti-viral” is used herein to refer to a composition which functions to increase cellular resistance to viral infection. The anti-viral effect of wild-type STAT1 is triggered in the presence of interferon, a chemical commonly produced by infected host cells. The activity of the present STAT1 mutant appears to be similarly triggered, and thus, is useful to treat infection by viruses which are generally inhibited by interferon, including both DNA and RNA viruses. Examples include, but are not limited to, viruses of the influenza family, herpesviruses including herpes simplex I and II, cytomegalovirus, Epstein-Barr virus, vaccinia virus, hepatitis viruses such as HepB and Hep C, encephalomyelcarditis virus, vesicular stomatitis virus, the various strains thereof and other viruses related thereto.

The term “anti-proliferative” as it is used herein is meant to refer to the decrease or prevention of cell proliferation, particularly with respect to the uncontrolled cellular proliferation that occurs in the formation of tumours. Without wishing to be limited to any particular mode of action, the STAT1 mutant appears to inhibit activated Stat3 proteins, which have recently been confirmed as having a causal role in oncogenesis. Stat1 mutant has been found to heterodimerize with activated (i.e. tyrosine phosphorylated) Stat3. Significantly, a higher amount of Stat3 binds to the Stat1 mutant than endogenous STAT1 in response to IFNs. These data substantiate previous findings for a functional cross-talk between the Stat1 and Stat3 proteins and provide evidence that the anti-proliferative properties of the Stat1 mutant are mediated, at least in part, by modulating Stat3 activity.

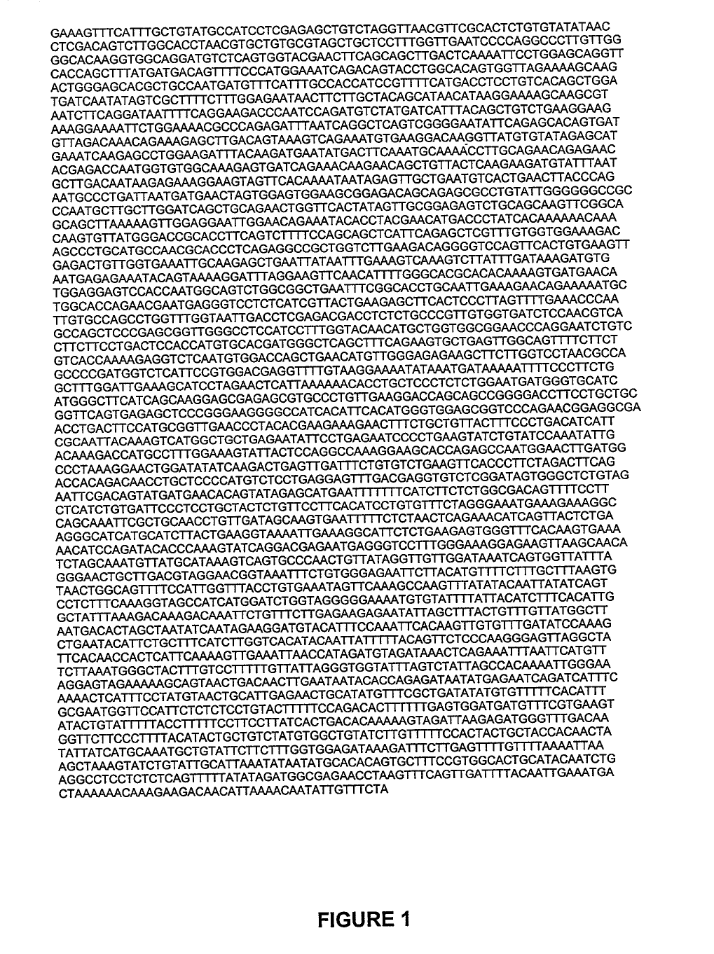

The STAT1 mutant results from an alteration in the sequence of the PKR binding region of STAT1, the sequence of STAT1 being encoded by the DNA sequence of FIG. 1. In one embodiment, the STAT1 mutant is altered such that PKR binding is abolished, or at least reduced. Accordingly, a preferred STAT1 mutant in accordance with the present invention is altered in the PKR binding region and, thus, comprises an altered amino acid sequence within positions 343-348 which results in a mutant having at least anti-viral activity. Although not wishing to be bound to any particular theory, it is believed that the activity of the mutant may, in part, be attributed to the lack of inhibition by PKR binding, as well as to a conformational change in protein structure that has resulted in a “gain of function”. In one preferred embodiment of the present invention, amino acids 346-348 are altered. A specific example of a change in amino acids 346-348 which renders a STAT1 mutant in accordance with the present invention is a change from Arg³⁴⁸-Leu³⁴⁷-Leu³⁴⁸ to Ala³⁴⁶-Asp³⁴⁷-Asp³⁴⁸. However, one of skill in the art will appreciate that similar modifications may be made in this particular region, for example, using similarly charged amino acids, to yield an anti-viral STAT1 mutant that does not bind PKR. Moreover, modifications to the sequence in the regions adjacent to amino acids 346-348 of STAT1 which are involved in PKR binding may also be made to yield an anti-viral STAT1 mutant which does not bind PKR.

It will be appreciated by those of skill in the art, that further modifications may be made to the STAT1 mutant to enhance its activity as an anti-viral agent which do not adversely affect this activity. For example, the mutant may be shortened in length to render it more readily useful in a pharmaceutical sense. Moreover, modification of side-chain moieties in a manner well-established in the art may also be to the STAT1 mutant in order particularly to enhance stability. Such modifications include the use of groups which protect the side-chain moieties from reaction or degradation. Modifications to the C- and N- terminal groups of the STAT1 mutant may also be made in order to prevent biological or chemical degradation thereof, thereby also increasing stability of the STAT1 mutant. Modifications of this nature are also well-known to those of skill in the art.

The advantageous effect of the STAT1 mutant may be obtained when administered as DNA, i.e. used in gene therapy, or when administered as a protein. Administration in either form is useful to enhance the anti-viral and anti-proliferative effects of cellular or exogenous interferon.

For use in methods of gene therapy, STAT1 mutant-encoding DNA may be derived from STAT1-encoding DNA, the sequence of which is set out FIG. 1. For this purpose, STAT1-encoding DNA is obtained from appropriate human cDNA libraries by screening with a STAT1 antibody or appropriate nucleic acid probe as would be appreciated by those of skill in the art. Once STAT1-encoding DNA is obtained, site-directed mutagenesis, as described by Kunkel et al. in Proc. Natl. Acad. Sci., 1985, 82:488 and also in the specific examples included herein, may be used to alter the DNA to encode the STAT1 mutant of interest. In general terms, site-directed mutagenesis is used to alter specifically one or more amino acids in a given peptide sequence by mutating specific nucleotide bases in the DNA sequence encoding the peptide. Thus, a segment of DNA encoding the region of STAT1 to be altered is cloned into a bacteriophage vector, such as an M13 bacteriophage vector. Replication of the vector in the phage results in single-stranded DNA that serves as a template for the hybridization of a primer oligonucleotide. The primer complements the DNA template with the exception of the nucleotide base changes required to render the desired mutant. The primer anneals to the single-stranded template and replication to form double-stranded DNA occurs, which ultimately yields single-stranded DNA, half of which is native STAT1-encoding DNA and the other half of which is mutated STAT1-encoding DNA. DNA encoding mutated STAT1 may be selected for using any one of several -selection techniques known in the art, for example, a radiolabelled oligonucleotide probe may used for selection purposes. DNA obtained in this way may be amplified using the polymerase chain reaction (PCR) technique.

Therapeutic treatment by gene therapy involves the transfer and stable insertion of new genetic information into cells. Having obtained STAT1 mutant-encoding DNA, its introduction into a progenitor cell or any other appropriate cell requires a method of gene transfer to integrate the STAT1 mutant-encoding DNA into the cellular genome. This can be done using viral vector systems, such as oncoretroviral vectors, adenovirus vectors and adeno-associated virus vectors, or by using nonviral vector systems such as liposomes, direct injection/particle bombardment and receptor-mediated endocytosis.

For the use of a viral vector system encoding STAT1 mutant for anti-viral and/or anti-proliferative treatment, STAT1 mutant-encoding DNA is first incorporated into an appropriately selected vector. Transduction of appropriate host cells with the vector DNA is then accomplished using standard methods. Host cells appropriate for transduction are obtained from the patient, i.e. mammal, using methods familiar to those of skill in the art of gene therapy. Alternatively, transduction is accomplished by infection of host cells with mature virions containing the vectors of interest. In this regard, the host cells are infected with the mature virions for about 1-2 hours at a temperature of about 37 degrees C. Stable integration of the viral genome is accomplished by incubation of HSC at about 37 degrees C. for about one week to about one month. Stable, site-specific integration and expression is assessed to confirm transduction by conducting an assay specific for the product, i.e. STAT1 mutant, or by conducting an assay for a marker associated with the product, for example, an antibody, in a manner well-established in the art. Transduced cells are then introduced into the patient requiring treatment, e.g. by intravenous transfusion (see, for example, Rosenberg, 1990). Again, presence of the STAT1 product in the patient can be monitored using the appropriate assay as set out above.

Nonviral vector system may also be used to deliver STAT1 mutant-encoding DNA to a mammalian patient. For example, the DNA may be packaged into liposomes which are delivered to a suitable target tissue in the patient. Alternatively, the DNA can be directly injected into a specific tissue of the patient, such as muscle. Particle bombardment is another direct injection approach in which the DNA is coated onto metal pellets and “fired” into cells using a special gun. Another method of introducing the DNA is receptor-mediated endocytosis in which the DNA is coupled to a target molecule that binds to a specific cell surface receptor, inducing endocytosis and transfer of DNA into the cells.

In an alternative to introducing STAT1 mutant-encoding DNA as a treatment protocol in accordance with the present invention, STAT1 mutant protein per se may be administered to a mammal in need of treatment. In this regard, STAT1 mutant can, of course, be prepared using recombinant technology given STAT1 mutant-encoding DNA prepared as described above. Methods employed in this regard are well established. Generally, this technology involves incorporation of DNA encoding the desired STAT1 mutant protein into an organism via a vector, culturing the organism and isolating and purifying the expressed product.

The STAT1 mutant protein is administered to a patient in combination with at least one pharmaceutically acceptable carrier that is suitable for combination with protein-based drugs. By “pharmaceutically acceptable” carrier is meant a carrier that is non-toxic and acceptable for use in the pharmaceutical and veterinary arts. Commonly used carriers include diluents, excipients and the like which facilitate administration. Examples of carriers which may be combined with the STAT1 mutant protein to form a composition suitable for oral administration include, but are not limited to, sugars, starches, cellulose and derivatives thereof, wetting agents, lubricants such as sodium lauryl sulfate, stabilizers, tabletting agents, colouring agents and flavouring agents. Reference may be made to “Remington's Pharmaceutical Sciences”, 17th Ed., Mack Publishing Company, Easton, Pa., 1985, for other carriers that would be suitable for combination with the present STAT1 mutant to render an orally ingestible composition. Carriers which may be combined with the STAT1 mutant protein to form a composition suitable for administration by injection include, for example, liquid adjuvants such as buffered saline and physiological saline. As will be appreciated, the pharmaceutical carriers used to prepare compositions in accordance with the present invention will depend on the administrable form to be used. Accordingly, for administration of the STAT1 mutant as a cream, ointment or lotion, carriers suitable to render such a composition would be used. Likewise, for administration of the STAT1 mutant in spray form, the mutant would be combined with carriers appropriate to render such a composition. Other adjuvants may also be added to the composition regardless of how it is to be administered, for example, preservatives, anti-microbial agents and anti-oxidants, to prolong the shelf life thereof.

In another of its aspects, the present invention provides a method of treating viral infection in a patient. The method comprises administration of a “therapeutically effective amount” of STAT1 mutant protein to a mammal, including both human and non-human mammals, which may require treatment. By “therapeutically effective amount” is meant an amount of STAT1 mutant protein that will result in an anti-viral effect that is greater than the anti-viral effect of interferon alone, while not exceeding an amount which may cause significant adverse effects. In this regard, precise dosage sizes can readily be established in appropriately controlled trials.

In another aspect of the present invention, there is provided a method for treating conditions associated with uncontrolled proliferation of cells in a mammal. Such conditions may result in the growth of either malignant tumours, for example, carcinoma, lymphoma and sarcoma, or benign tumors.

The method involves administration to the mammal of a therapeutically effective amount of STAT1 mutant protein. In this instance, “therapeutically effective amount” is meant to encompass an amount of STAT1 mutant protein that will result in an anti-proliferative effect that is greater than the anti-proliferative effect of interferon alone, while not exceeding an amount which may cause significant adverse effects. For anti-proliferative treatment, administration of a STAT1 mutant protein, administered either in the form of DNA or in protein form, can be readily confirmed by standard trials as would be appreciated by one of skill in the art.

The anti-viral and anti-proliferative effects provided in the presence of a STAT1 mutant in accordance with the present invention may be further enhanced when administered in conjunction with other anti-viral or anti-proliferative agents. Examples of anti-viral agents that would be useful in conjunction with a STAT1 mutant in accordance with the present invention include interferons. Examples of anti-proliferative agents for use in conjunction with STAT1 mutant include interferons and TGF-β. Such a combination therapy may involve administration of discrete compositions or co-administration of therapeutics. As noted above, such compositions will be prepared with a pharmaceutically acceptable carrier selected for its suitability depending on the route of administration.

Embodiments of the present invention are described in the following specific examples which are not to be construed as limiting.

### EXAMPLE 1

### Determination of STAT1 Binding Site to PKR

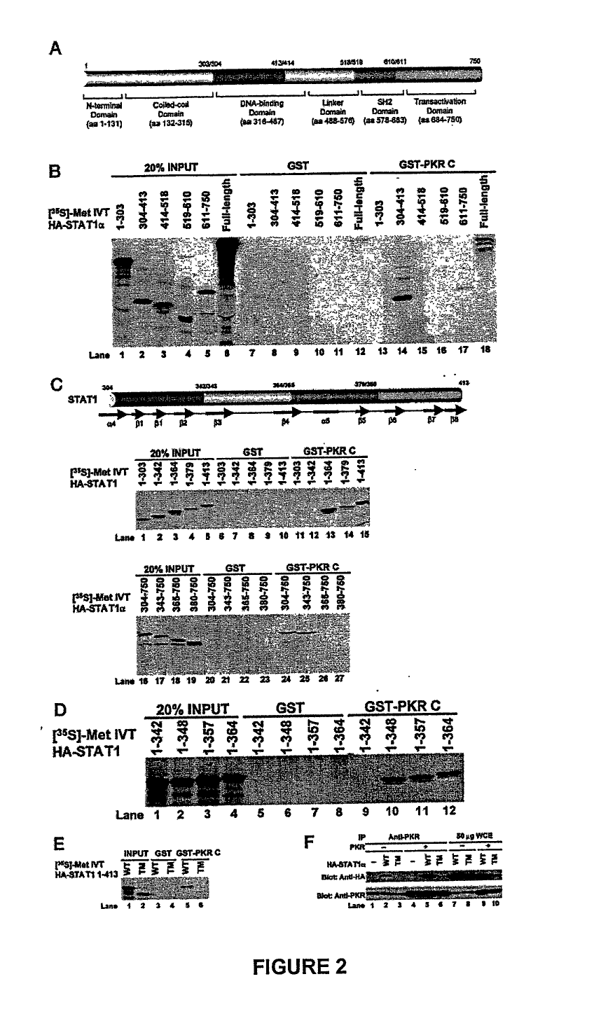

To map the region on STAT1 that facilitates its interaction with PKR, truncated STAT1 proteins corresponding to amino-terminal, DNA-binding, linker, SH2, or transactivation domains were used (FIG. 2A). The pull-down assays were performed with GST-PKR C, because this protein binds to STAT1 and is more stable than GST-PKRK296R (data not shown). GST-PKR C specifically interacted with the DNA-binding domain of STAT1 (FIG. 2B, lane 14). Upon truncation of the STAT1 DNA-binding domain from the carboxyl-terminal end, a critical junction is reached when binding to PKR is retained (middle panel, HA-STAT1 1-364, lane 13) and when binding is abolished (HA-STAT1 1-342, lane 12). Further truncation of STAT1 from the amino terminus also presented a similar junction between amino acids 343-365 (FIG. 2C, lower panel, compare lanes 25 and 26). Moreover, truncation of the carboxyl terminus of the DNA-binding domain of STAT1 at positions 348 and 357 still retained binding to GST-PKR (FIG. 2D, lanes 10 and 11), suggesting that the region of interaction lies between amino acids 343 and 348 (β-sheet 3) on the DNA-binding domain of STAT1 (Green, S. R., and Mathews, M. B. (1992) Genes Dev. 6, 2478-2490; Chen, X., vinkemeier, U., Zhao, Y., Jeruzalmi, D., Darnell, J. E. J., and Kuriyan, J. (1998) Cell 93, 827-839).

### EXAMPLE 2

### Preparation of a STAT1 Mutant Unable to Bind PKR

To identify amino acids in STAT1 that form contacts with PKR, mutations were constructed within amino acids 343-348 of HA-STAT1 1-413 that abolished binding to PKR. Alanine-scan mutagenesis of each of the six amino acids did not yield a point mutant of HA-STAT1 1-413 that disrupted interaction with PKR (data not shown). However, a three-amino acid substitution (TM or Mutant; Arg³⁴⁶-Leu³⁴⁷-Leu³⁴⁸ to Ala³⁴⁶-Asp³⁴⁷-Asp³⁴⁸) within this region disrupted the ability of STAT1 to interact with GST-PKR C (FIG. 2E, lane 6). Interestingly, HA-STAT1 1-413 TM possessed faster mobility on SDS-PAGE gels compared with WT most likely through changes in the overall charge of the molecule.

To further characterize the interaction of full-length HA-STAT1α TM with PKR, the human fibrosarcoma, U3A cell line was utlized, which lacks endogenous STAT1 (McKendry, R., John, J., Flavell, D., Muller, M., Kerr, I. M., and Stark, G. R. (1991) Proc. Natl. Acad. Sci. U.S.A. 88, 11455-11459). HA-STAT1α WT or TM were co-transfected with PKRK296R into U3A cells, after which, the protein extracts were immunoprecipitated against PKR and immunoblotted with HA antibodies. As seen in FIG. 2F, STAT1α WT associated with both transfected and endogenous PKR (upper panel, lanes 2 and 5). Conversely, STAT1 TM binding with endogenous PKR was completely abolished (lane 3) and displayed very marginal binding to transfected PKR, which was detectable only after long exposures (lane 6). In contrast to HA-STAT1 1-413 TM, full-length STAT1α TM did not display any difference in its migration pattern compared with STAT1α WT.

These in vitro findings were also verified in vivo by the yeast two-hybrid assay.

Taken together, it appears that the DNA-binding domain at STAT1 interacts with PKR in vitro and in vivo.

### EXAMPLE 3

### Properties of the STAT1 Mutant

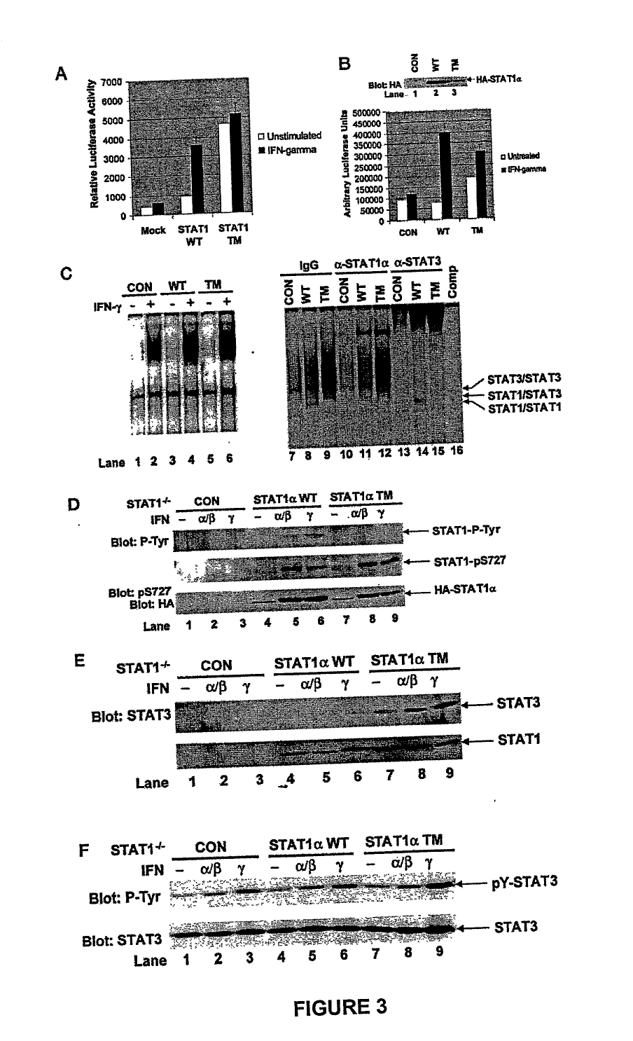

DNA Binding and Transcriptional Properties of STAT1 TM—To test the ability of STAT1 TM to respond to IFN-y treatment, transient transfection assays were performed in STAT1^{-/-} cells using STAT1 WT or STAT1 TM and a luciferase reporter construct driven by two copies of the GAS element from the IFP53 gene (Eilers, A., Baccarini, M., Horn, F., Hipskind, R. A., Schindler, C., and Decker, T. (1994) Mil. Cell. Biol. 14, 1364-1373). As shown in FIG. 3A, luciferase expression in cells transfected with STAT1 WT was induced by IFN-y treatment. However, in cells transfected with STAT1 TM, after normalization to Renilla luciferase, a much higher basal luciferase activity (˜5-fold) was observed that could be slightly induced by IFN-γ stimulation.

To better characterize STAT1 TM, STAT1^{-/-} fibroblasts were infected with retroviruses harboring the puromycin-resistant gene and HA-STAT1α WT or HA-STAT1α TM. As a control, retroviruses containing only the puromycin-resistant gene were used. After puromycin selection, polyclonal populations of STAT1 WT-expressing cells showed ˜5-fold greater expression over STAT1 TM pools (FIG. 3B, compare lanes 2 and 3). Transactivation assays using the 2XIFP53-GAS luciferase reporter correlated with findings in transient transfection experiments that STAT1 TM confers higher basal reporter activity, which can be induced by IFN-γ treatment (FIG. 3B). STAT1 TM DNA binding following IFN treatment was tested. Although ISGF3 formation in STAT1 TM cells could not be detected in response to IFN-α/β (data not shown), IFN-γ stimulation resulted in the formation of DNA-binding complexes consisting of either STAT1.STAT3 heterodimers or STAT3 homodimers, but not that of STAT1 homodimers (FIG. 3C, compare lanes 7-16). This finding is consistent with previous reports that STAT3 is activated following IFN treatment (3). Moreover, the intensity of the STAT3 homodimer appears to be higher compared with control or STAT1 WT cells (compare lanes 2, 4, and 6).

The ability of STAT1 to be phosphorylated upon IFN stimulation was also examined (FIG. 3D). To compare STAT1 phosphorylation per equal amounts of STAT1 protein, a 5-fold higher amount of STAT1 TM extracts versus STAT1 WT were used before and after IFN stimulation. These reactions were also normalized to total protein concentration by the addition of treated or untreated STAT1^{-/-} control extracts. Although STAT1 WT was tyrosine-phosphorylated following IFN-α/β or IFN-γ treatment (top panel, lanes 5 and 6), STAT1 TM tyrosine phosphorylation was not detected (lanes 8 and 9). In contrast, phosphorylation of serine 727 did not significantly differ between STAT1 WT and STAT1 TM after IFN treatment (middle panel, lanes 5-6 and 8-9). Reprobing of the membrane with antibodies to HA revealed the hypo- and hyperphosphorylated forms of STAT1 usually observed after IFN treatment (lower panel). Because STAT1 is also known to form heterodimers with STAT3 following IFN stimulation (Darnell, J. E., Jr. (1997) Science 277, 1630-1635), STAT1 TM association with STAT3 was tested. A much higher amount of STAT3 co-precipitated with STAT1 TM before and after IFN treatment (FIG. 3E, top panel, lanes 7-9), although STAT1 protein levels were approximately equal (bottom panel). Expression and activation of STAT3 was also analyzed in the same protein extracts used for STAT1/STAT3 co-immunoprecipitation. STAT3 phosphorylation was slightly elevated (˜50%) in cells expressing STAT1 TM before or after treatment with either IFN-α/β or IFN-γ (FIG. 3F, lanes 7-9). This increase in STAT3 activity may account for increased STAT3 DNA binding in STAT1 TM cells.

### EXAMPLE 4

### Enhanced Anti-viral and Anti-proliferative Effects of a STAT1 Mutant

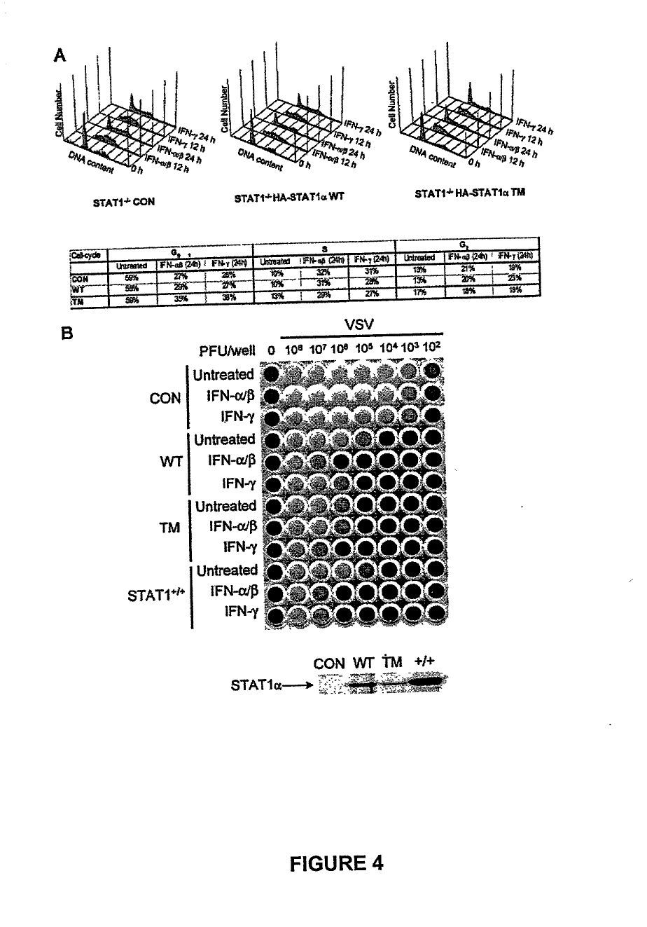

The biological effects of STAT1 TM activation were examined by cell cycle analysis after treatment with IFN-α/β or IFN-γ (FIG. 4A). A greater proportion of STAT1 TM-expressing cells (IFN-α/β, 6-8%; IFN-γ, 10-11%) were arrested in G₀/G₁ phase after treatment with either type I or type II IFNs (right panel) compared with control (left panel) or STAT1 WT-expressing (middle panel) cells. In addition, the ability of STAT1 TM cells to resist virus infection was also investigated. Control, STAT1 WT, and STAT1 TM cells were primed with IFNs and subsequently infected with serially diluted VSV. The amount of virus needed to induce CPE was qualitatively measured. As shown in FIG. 4B (upper panel), STAT1 TM cells that were treated with IFN-γ were ˜50-fold more resistant to VSV infection compared with STAT1 WT cells, and ˜10⁴-fold more resistant versus control STAT1^{-/-} cells. In contrast, IFN-α/β-treated STAT1 TM cells were 10-fold more susceptible to VSV CPE compared with control, STAT1 WT, and STAT1^{+/+} cells. Interestingly, even untreated STAT1 TM cells provided a greater degree of protection compared with STAT1 WT and STAT1^{+/+} cells.

This enhanced ability of STAT1 TM cells to resist virus infection was also observed after encephalomyelocarditis virus infection.

Western blotting against STAT1α revealed that STAT1 TM is expressed at much lower levels than STAT1 WT and endogenous STAT1 from STAT1^{+/+} cells (bottom panel). Taken together, these data suggest that STAT1 TM enhances the antiproliferative and antiviral effects of IFNs on a per molecule basis.

### EXAMPLE 6

### Activity of a STAT1 Mutant in vivo

The first step in producing transgenic animals is to construct the DNA to be transferred (i.e. the transgene) Babinet, C. (2000) J. Am. Soc. Nephrol. Suppl. 16: S88-94. A critical component of the transgene is the promoter, the regulatory region that drives transcription. In this case, ubiquitous expression of STAT1 TM is desired. However, the growth inhibitory effects mediated by the constitutive expression Stat1 TM in cultured cells may prove detrimental during mouse development and impair the analysis of the phenotype in the adult animal. To bypass this limitation, a system that allows the inducible expression of Stat1 TM has been chosen. To date, two major inducible systems have been successfully used in transgenic mice: The tetracycline (Tet)-inducible system and the Cre/loxP recombinase system Jaisser, F., (2000) J. Am. Soc. Nephrol. Suppl. 16: S95-100. To use these systems in vivo, it is necessary to generate two sets of transgenic animals. One mouse line expresses the “activator” (tTA, rtTA, or Cre recombinase) under the control of a selected tissue-specific promoter. Another set of transgenic animals expresses the “acceptor” construct, in which the expression of the transgene is under the control of the target promoter for the tTA/rtTA transactivators or is flanked by loxP sequences. Mating the two strains of mice allows spatiotemporal control of transgene expression.

The need for two sets of transgenic animals has been recently bypassed by the development of a modified Tet-inducible system by Holzenberger and colleagues Holzenberger, M., et al. (2000) Genesis 26:157-159. The Tet systems in general permit stringent control of gene expression in a wide range of cells in culture and in transgenic animals Jaisser, F. (2000) J. Am. Soc. Nephrol. Suppl. 16: S95-100. It relies on two components, i.e. a tet-controlled transactivator (tTA or rtTA) and tTA/rtTA-dependent promoter that control the expression of the downstream cDNA (e.g. Stat1 TM cDNA), in a tetracycline-dependent manner. In the absence of tetracycline or its derivatives (e.g. doxycycline), tTA binds to the promoter, allowing transcriptional activation of the gene. The tet system using tTA is termed tet-OFF, because tetracycline or doxycycline permits transcriptional downregulation. A mutant form of tTA, termed rtTA, is not functional in the absence of doxycycline but requires the presence of the ligand for transactivation. This system, termed tet-ON, has the advantage over tet-OFF that the transgene is not expressed until doxycycline is given to the animals and that upregulation in vivo is faster than downregulation.

The system designed by Holzenberger et al. Holzenberger, M., et al., (2000) Genesis 26:157-159 is tet-ON and uses a doxycycline auto-inducible (DAI) single construct, which contains both rtTA and the gene of interest under the control of the same bi-directional doxycycline responsive promoter. This system bypasses the need of “activator” and “acceptor” mouse strains required for the establishment of the conventional tetracycline-inducible or Cre/lox transgenics. Moreover, the bi-directional tet-ON system bypasses mosaic transgene expression and the toxic effects of the constitutive expression of the rtTA transactivator reported with the conventional tet-ON system. The DAI construct has been obtained and further modified it by subcloning the Stat1 TM cDNA, which contains the hemagglutinin HA epitope tag in the 5′ end of the gene, together with the lacZ reporter DNA downstream of the tetracycline-inducible promoter. The construct has been engineered so that the HA-Stat1 TM and lacZ genes are expressed from the same bi-cistronic RNA, which contains an internal ribosomal entry site (IRES) between the two genes (downstream of HA-Stat1 TM and upstream of lacZ). The presence of the HA tag in Stat1 TM allows the detection of the mutant protein from endogenous mouse Stat1 by immunoblotting with anti-HA antibodies whereas the presence of lacZ facilitates the screening of pups expressing the Stat1 TM transgene and the detection of transgene expression by lacZ/X-gal histology on frozen sections of different tissues after induction with doxycycline.

The introduction of HA-Stat1 TM transgene into the genome requires fertilized mouse eggs, Hogan, B., et al. (1994) Manipulating the Mouse Embryo: A Laboratory Manual. Cold Spring Harbor Laboratory Press, U.S.A. Injection of gonadotropins (typically, a mixture of pregnant-mare serum gonadotropin and human chorionic gonadotropin) into a female mouse of FVB/NHSD genetic background will induce hyperovulation, which, followed by natural mating with a fertile male, will provide the source of eggs. The fertilized eggs will be harvested before the first cleavage and placed into a petri dish. The HA-Stat1 TM/lacZ transgene (about 100 to 200 copies in 2 pl of buffer) will be introduced by microinjection through a fine glass needle into the male pronucleus (the nucleus provided by the sperm before fusion with the nucleus of the egg). These manipulations are performed with a binocular microscope at magnification of ×200. The injected eggs will then be cultured to the two-cell stage and then implanted into oviducts of recipient pseudopregnant females. In these types of experiments, a total of 25 to 30 injected embryos are usually implanted. After 19 to 20 days of gestation, pups are born. Typically, 15 to 30% of the injected embryos will proceed to term, and 10 to 20% of these full term embryos will have integrated the transgene into their germ-line DNA. The transgenic pups, called founders, will be identified by testing their genomic DNA, usually obtained from the tails of the pups, for the HA-STAT1 TM/lacZ transgene by southern blot analysis or the polymerase chain reaction (PCR). Typically, 1 to 200 copies of the transgene are incorporated in a head-to-tail orientation into a single random site in the mouse genome. Since injection and integration occur before the first cell division, all cells of the founders, including the germ cells, will be heterozygous for the STAT1 TM/lacZ transgene.

Once founder mice are determined to be transgenic, they will be mated with mice from the same inbred strain to begin establishing STAT1 TM transgenic lines. Female founder mice are usually kept until they have given birth and raised one litter before they are sacrificed. Male founder mice are not sacrificed until positive transgenic progenies are identified. Once the establishment of transgenic lines is ensured, expression of STAT1 TM in founder mice will be induced by the presence of doxycycline in the drinking water of the animals and visualized by LacA/X-gal histology in different tissues of sacrificed animals or by immunostaining with anti-HA antibodies. Mating of heterozygous mice from the same transgenic line will generate permanent homozygous lines that are used in the phenotype analysis after virus infection. That is, STAT1 TM transgenic mice are tested for their susceptibility to infection with a variety of viruses such as Newcastle disease virus (NDV), vesicular stomatitis virus (VSV), encephalomyelocarditis (EMCV), Sendai and influenza virus at various multiplicities of infection (m.o.i.), as has been previously described for other Stat transgenic mice Durbin et al., (1996) Cell 84: 443-450; Meraz et al., (1996) Cell 84: 431-442; and Park, C., et al., (2000) Immunity 13: 795-804.

METHODS AND MATERIALS

Cell Culture and Transfections—HeLa S3, U3A, STAT1^{+/+}, STAT1^{-/-}, PKR^{+/+}, and PKR^{-/-} cells were maintained in Dulbecco's modification of Eagle's medium supplemented with 10% calf serum, 2 mM L-glutamine, and 100 units/ml penicillin/streptomycin (Life Technologies). For IFN treatment, cells were incubated with 1000 IU/ml of recombinant murine IFN-α/β (Lee Biomolecules) or 100 IU/ml of IFN-y (PharMingen). Double-stranded RNA transfections were performed as previously described (Wong, A. H., et al. (1997) EMBO J. 16, 1291-1304). Transient transfections were performed with LipofectAMINE Plus reagent (Life Technologies). T7 vaccinia virus transient transfections were carried out using the recombinant vaccinia virus, vTF7-3, encoding the bacteriophage T7 RNA polymerase (Fuerst, T. R., et al. (1986) Proc. Natl. Acad. Sci. U.S.A. 83, 8122-8126). In vivo [³⁵ S]methionine experiments were performed as previously described (Tam, N. W., et al. (1999) Eur. J. Biochem. 262, 149-154). pBABE, pBABE-HA-STAT1a WT, or TM STAT1^{-/-} cells were generated as previously described (Morgenstern, J. P., et al. (1990) Nucleic Acids Res. 18, 3587-3596). Cell cycle analyses and CPE assays were performed as described in previous studies (Cuddihy, A. R., et al. (1999) Mol. Cell. Biol. 19, 2475-2484; Garcia-Sastre, A., et al. (1998) J. Virol. 72, 8550-8558).

Plasmid Construction—GST-PKRK296R, GST-PKR 1-262 (PKR N), and GST-PKR K296R 263-551 (PKR C) were generated as previously described (Cuddihy, A. R., et al. (1999) Oncogene 18, 2690-2702). GST-PKRLS4K296R was generated by site-directed mutagenesis using the QuikChange site-directed mutagenesis kit (Stratagene) with the following primer pairs: (Sheldon Biotechnology): 5′-d[CCAGAAGGTGAAGGTGGAGCATTGAAGGAAGCAAAA AATGC-CGC]-3′ and 5′-d[GCGGCATTTTTTGCTTCCTTCAATGCTCCACCTTCACCT TCTGG] -3′. GST-PKRLS9K296R was generated by subcloning an EcoNI and AflII fragment of PKRLS9. Truncated GST-PKR proteins were generated with the following primers: pGEX forward primer (Amersham Pharmacia Biotech); 5′-PKR 263, 5′-d[GG GGGATCCTAAAACCTCTTGTCCACAGTATAC]-3′; 5′-PKR 325, 5′-d[GGGGGGATC CGGCTGTTGGGATGGATTTGATTAT]-3′; 5′-PKR 367, 5′-d[GGGGGGATCCTTCTG TGATAAAGGGACCTTGGAA]-3′; 3′-PKR 262, 5′-d[GGGGGATCCTAAAACCTCTTGT CCACAGTATAC]-3′; 3′-PKR 324, 5′-d[CCCGGGCTAATTGTAGTGAACAATATTTA CATGAT]-3′; 3′-PKR 366, 5′-d[CCCGGGCTATTCCATTTGGATGAAAAGGCACT]-3; 3′-PKR 415, 5′-d[CCCGGGCTACTTAAGATCTCTATGAATTAATTTTTT]-3′; and pGEX reverse primer (Amersham Pharmacia Biotech). PCR fragments were cut with BamHI and/or SmaI and ligated to pGEX-2T. Truncated STAT1 proteins were generated by PCR from the template pGEX-5X-3-HA-STAT1 (26) using the following primers: 5′-HA-STAT1, 5′-d[GGGGGGATCCACCATGGCATACCCATACGACGTCCCAGATTACGCT ATGTCTCAGTGGTACGAACTTCAG]-3′; 5′-HA-STAT1 304, 5′-d[GGGGGGATCCACC ATGGCATACCCATACGACGTCCCAGATTACGCTCGCACCTTCAGTCTTTTCCAG]-3′; 5′-HA-STAT1 343, 5′-d[GGGGGGATCCACCATGGCATACCCATACGACGTCCC AGATTACGCTGTGAAGTTGAGACTGTTGGTGAAA]-3′; 5′-HA-STAT1 365, 5′-d[GGGGGGATCCACCATGGCATACCCATACGACGTCCCAGATTACGCTGATAAAGATG TGAATGAGAGAAATAC]-3′; 5′-HA-STAT1 380, 5′-d[GGGGGGATCCACCATGGCA TACCCATACGACGTCCCAGATTACGCTTTCAACATTTTGGGCACGCACAC]-3′; 5′-HA-STAT1 414, 5′-d[GGGGGGATCCACCATGGCATACCCATACGACGTCCCAGA TTACGCTAATGCTGGCACCAGAACGAATG]-3″; 5′-HA-STAT1 519, 5′-d[GGGGGGA TCCACCATGGCATACCCATACGACGTCCCAGATTACGCTCTGAACATGTTGGGAG AGAAGC]-3′; 5′-HA-STAT1 611, 5′-d[GGGGGGATCCACCATGGCATACCCATACG ACGTCCCAGATTACGCTGCCATCACATTCACATGGGTG]-3′; 3′-STAT1 303, 5′-d [GGG-GCGGCCGCCTAGTCCCATAACACTTGTTTGTTTTT]-3′; 3′-STAT1 342, 5′-d[GGGGCGGCCGCCTAAGTGAACTGGACCCCTGTCTTC]-3′; 3′-STAT1 348, 5′-d[GGGGCGGCCGCCTACAACAGTCTCAACTTCACAGTGAA]-3′; 3′-STAT1 357, 5′-d[GGGGCGGCCGCCTAATTATAATTCAGCTCTTGCAATTTCA]-3′; 3′-STAT1 364, 5′-d[GGGGCGGCCGCCTAAAATAAGACTTTGACTTTCAAATTATAAT]-3′; 3′-STAT1 379, 5′-d[GGGGCGGCCGCCTACTTCCTAAATCCTTTTACTGTATTTC]-3′; 3′-STAT1 413, 5′-d[GGGGCGGCCGCCTATTTCTGTTCTTTCAATTGCAGGTG]-3′; 3′-STAT1 518, 5′-d[GGGGCGGCCGCCTACTGGTCCACATTGAGACCTCT]-3′; 3′-STAT1 610, 5′-d[GGGGCGGCCGCCTACCCTTCCCGGGAGCTCTCA]-3′; and the pGEX reverse primer. PCR products were BamHI-NotI-digested, subcloned into the mammalian expression vector, pcDNA3.1/zeo, and confirmed by sequencing. Site-directed mutagenesis was carried out with the following primer pairs: R346A, 5′-d[CAGTTCACTGTGAAGTTGGCACTGTTGGTGAAATTGCAAG]-3′ and 5′-[CTTGCAATTTCACCAACAGTGCCAACTTCACAGTGAACTG]-3′; R346A/L347D, 5′-[CAGTTCACTGTGAAGTTGGCAGACTTGGTGAAATTGCAAGAGCTG]-3′ and 5′-[CAGCTCTTGCAATTTCACCAAGTCTGCCAACTTCACAGTGAACTG]-3′; and R346A-/L347D/L348D, 5′-[GTTCACTGTGAAGTTGGCAGACGACGTGAAATTGCAAGAGCT G]-3′ and 5′-[CAGCTCTTGCAATTTCACGTCGTCTGCCAACTTCACAGTGAAC]-3′. PCR products were ligated to pcDNA3.1/zeo-HA-STAT1 by EcoNI restriction digest. pBABE- HA-STAT1aWT and TM were generated by ligation into BamHI restriction sites.

Cell Extract Preparation, Immunoprecipitation, and Immunoblot Analysis—Cell extract preparation, immunoprecipitation, and immu-noblotting were performed as previously described (Wong, A. H., et al. (1997) EMBO J. 16, 1291-1304). The following antibodies were used: STAT1a (Santa Cruz Biotechnology); HA (12CA5, Roche Molecular Biochemicals); STAT2 (Upstate Biotechnology Inc.); STAT3 (Santa Cruz); PKR; GST (Amersham Pharmacia Biotech); Myc (9E10, Roche Molecular Biochemicals); eIF-2a; phosphoserine 51 of eIF-2a (Research Genetics Inc.); FLAG (M2, Kodak); HA horseradish peroxidase antibody (3F10, Roche Molecular Biochemicals); phosphotyrosine (4G10/PY20, UBI and Transduction Laboratories); and phosphoserine 727 of STAT1a (Kovarik, P., et al. (1998) EMBO J. 17, 3660-3668). Proteins were visualized by ECL (Amer-sham Pharmacia Biotech).

DNA Binding and Transactivation Assays—Electrophoretic mobility shift analysis was performed using the dsDNA c-Fos c-sis-inducible element (SIE, 5′-GATCGTGCATTTCCCGTAAATCTTGTCTACAATTC-3′) according to protocols previously described (Wong, A. H., et al. (1997) EMBO J. 16, 1291-1304; Eilers, A., et al. (1994) Mol. Cell. Biol. 14, 1364-1373). The Dual Luciferase system (Promega) was used to assess the transactivation potential of STAT1. Briefly, STAT1^{-/-} cells or cells expressing STAT1 WT or TM were transfected with Renilla luciferase (pRL-TK) and pGL2XIFP53 GAS luciferase. Twenty-four hours after transfection, cells were replated and treated with IFN-γ for 18 h before harvesting. The results presented represent quadruplicate experiments where GAS luciferase activity was normalized to Renilla luciferase activity.

Isoelectric Focusing and PKR in Vitro Kinase Assays—Isoelectric focusing and immunoblot analysis of yeast eIF-2a were performed as previously described (Kawagishi-Kobayashi, M., et al. (1997) Mol. Cell. Biol. 17, 4146-4158). PKR in vitro kinase assays were carried as previously described (Wong, A. H., et al. (1997) EMBO J. 16, 1291-1304).

GST Pull-down Assays—Protein production and extraction were performed according to previously described protocols (Cuddihy, A. R., et al. (1999) Oncogene 18, 2690-2702; Zhang, J. J., et al. (1996) Proc. Natl. Sci. U.S.A. 93, 15092-15096). Normalized GST fusion proteins were co-incubated with HeLa whole cell lysates or [³⁵S]methionine in vitro translated proteins, washed, subjected to SDS-PAGE, and visualized by fluorography (Cuddihy, A. R., et al. (1999) Oncogene 18, 2690-2702).

Yeast Plasmids, Transformations, Growth Protocols, and Protein Ex-tractions—Wild-type and mutants of HA-STAT1 1-413 were subcloned by restriction digest of BamHI-NotI sites into a modified form of the yeast expression vector, pEMBL/yex4 (Kawagishi-Kobayashi, M., et al. (1997) Mil. Cell. Biol. 17, 4146-4158), containing a NotI site in the multiple cloning site. Transformation of yeast strains H2544 and J110 and growth analyses were performed as previously described (Dever, T. E. (1998) Methods Mol. Biol. 77, 167-178).

We claim:

1. An anti-viral STAT1 mutant.

2. A STAT1 mutant as defined in claim 1 which does not bind PKR.

3. A STAT1 mutant as defined in claim 2 in which at least one amino acid is modified in the PKR binding region.

4. A STAT1 mutant as defined in claim 3, wherein amino acids at positions 346-348 are modified.

5. A STAT1 mutant as defined in claim 4, having the modified amino acid sequence, Ala³⁴⁶-Asp³⁴⁷-Asp³⁴⁸

6. An anti-viral composition comprising an anti-viral STAT1 mutant in combination with a pharmaceutically acceptable carrier.

7. An anti-viral composition as defined in claim 6, wherein the STAT1 mutant does not bind PKR.

8. An anti-viral composition as defined in claim 7, wherein the STAT1 mutant comprises at least one amino acid that is modified in the PKR binding region.

9. An anti-viral composition as defined in claim 8, wherein amino acids at positions 346-348 of the STAT1 mutant are modified.

10. An anti-viral composition as defined in claim 9, wherein the STAT1 mutant has the modified amino acid sequence, Ala³⁴⁶-Asp³⁴⁷-Asp^{348.}

11. An anti-viral composition as defined in claim 6, in combination with another anti-viral agent.

12. A composition as defined in claim 11, wherein said anti-viral agent is interferon-y.

13. A composition as defined in claim 3, in a form suitable for oral administration.

14. A method of treating a viral infection in a mammal comprising the step of administering to the mammal a therapeutically effective amount of an anti-viral STAT1 mutant.

15. A method as defined in claim 14, wherein the STAT1 mutant is administered in the form of DNA.

16. A method as defined in claim 15, wherein said DNA is stably tranduced in cells of said mammal.

17. A method as defined in claim 14, wherein an anti-viral agent is administered in conjunction with the STAT1 mutant.

18. A method as defined in claim 17, wherein the anti-viral agent is interferon-y.

19. A method of treating a condition associated with the uncontrolled proliferation of cells in a mammal, comprising the step of administering to the mammal a therapeutically effective amount of a STAT1 mutant having anti-proliferative activity.
